(19) United States
(12) Patent Application Publication
(10) Pub. No.: US 2022/0031758 A1
(43) Pub. Date: Feb. 3, 2022

(54) USE OF ANIMAL ORIGINATED CORNEA MEMBRANE PRODUCTS IN TREATMENT OF EYE AND OUT OF EYE WOUNDS

(71) Applicant: Nebahat BULUT, Istanbul (TR)

(72) Inventor: Nebahat BULUT, Istanbul (TR)

(21) Appl. No.: 17/298,784

(22) PCT Filed: Nov. 28, 2019
(86) PCT No.: PCT/TR2019/051007

§ 371 Date: Jun. 1, 2021

(30) Foreign Application Priority Data
Dec. 3, 2018 (TR) 2018/18391

(51) Int. Cl. A61K 35/30; A61K 9/06; A61P 17/02; A61K 9/00

(52) U.S. Cl. CPC A61K 35/30; A61K 9/0048; A61P 17/02; A61K 9/06

(57) ABSTRACT

The present disclosure relates to use of animal originated cornea layer in treatment of eye and out of eye wounds. In line with the purpose, the cornea layer is prepared in serum, plate, suspension, cream, and homogenate forms treated with therapeutics.

### THE RELATED ART

The present disclosure is related to use of animal originated eye cornea layer in treatment of eye and out of eye wounds.

The present disclosure is particularly related to transformation of animal originated cornea into various application forms in order to provide use in eye and out of eye, skin, mucosa and similar wet skin surface wounds, surgical cuts, skin ulcers, circulation disorder ulcers, diabetic wounds, cancer related wounds, chemical or burn wounds, bonds and tendon wounds in joints, military, sportive injures, intra oral wounds, sick bed wounds, wounds in anus area, post-surgical wounds and tissue deficiency treatments and cosmetic skin rejuvenations.

### BACKGROUND OF THE RELATED ART

Cornea layer is the outer transparent part covering colourful section of eye. Cornea rooting from ectoderm layer in human body consists of five layers, namely, epithelial layer, Bowman layer, stroma, Descemet layer and inner endothelial layer and is not seen in any part of body other than eyes. Differently from other sections of the body, cornea heals quickly with leaving minimum scar tissue. Healing time for cornea injuries may vary from 24-48 hours to 6-8 days depending on status of wound. Veinless structure of cornea tissue provides a great advantage in healing process. Since it has no veins, vein gemmation seen in other tissues is not seen in cornea and as a result minimum scaring occurs. Thus tissue of transparent, colourless and ideal thickness before injury can be produced. However, in remaining parts of body swelling, concavity, colouring and similar undesired status in wound tissues occur.

Area of use of cornea today is limited to surgical applications of transplantation from human or animal to human. In vivo cornea layers obtained from cadaver or animal sources are covered onto injured human cornea as wound covering. Life of in vivo tissues used for such action is limited to one week. In case of advanced cornea injury cases, damaged cornea is entirely removed and replaced with cornea taken from cadaver. Other than transplantation of new cornea in place of injured or entirely damaged cornea, there is no study in the related art wherein cornea is used in non-ophthalmic areas.

On the other hand, in the current status, various tissue injuries and treatment methods developed for said injuries have an important place in health field. Examples of such wounding are wet skin surface wounds such as skin, mucosa wounds, surgical cuts, skin ulcers, circulation disorder ulcers, diabetic wounds, cancer related wounds, chemical or burn wounds, tissue and tendon wounding in joints, military, sportive wounding, skin rejuvenation processes, intra oral wounds, sick bed wounds, wounds in anus area, post-surgical wounds and scars, tissue repair requiring tissue deficiencies. Late healing of said wounds, not healing, getting deeper, getting infection are frequently experienced cases negatively affecting living quality. In addition to various chemical, biological medicine compositions to provide wound healing, dried or wet plate or fluid serum forms of human and/or animal originated tissues are also used to provide treatment. The most known of them is amniotic membrane. Amniotic membrane is obtained from the sac carrying infant during pregnancy. Said amniotic membrane develops from ectoderm layer of embryo similar to cornea and does not contain muscle cell, lymphatic and blood veins. In this respect, risk of tissue objection among transplanted tissues is at minimum level theoretically and is almost zero in practice. However, taking it from mother placenta causes various risks. Conditions of hospital of birth delivery and health state of mother are all highly important in respect to amnion membrane. For instance, if mother has hepatitis, AIDS, diabetics diseases or smokes, undesired cases may be encountered. Also when preparing amnion membrane, an undressing is made and tissue of 0.2 mm to 0.5 mm thickness is obtained. Mostly, bottom section called corium membrane remains on placenta necessarily. And this causes limited healing factors functioning from bottom to up in wound covers originating from amnion membrane and oedematous and worse healing since there will be no oedema preventive pump mechanism (Na—K-ATPase) in bottom endothelial layer. Whereas, cornea can be taken without no deformation in all layers, and in dissections containing certain layers if desired so, and corneal wound covers of different thickness can be prepared for wounds of different depth. At present, ophthalmologists can separate layers of any desired number when they take cornea layer or after.

When patent and literature search is made in the related art, no document disclosing use of dried and fluid forms of animal cornea for eye diseases has been seen. No document disclosing use of cornea tissue either in vivo or non in vivo form (dried or serum) for healing of out of eye wounds has been seen.

As a result, due to above described disadvantages and inadequacy of existing solutions it has been necessary to make a novelty and development in the related art.

### BRIEF DESCRIPTION OF THE INVENTION

The present invention relates to method for developing a wound healing material meeting the needs mentioned above, eliminating all disadvantages and providing some additional advantages.

The main purpose of the invention is to prepare a material healing various tissue wounds quickly and without leaving any scars. In the invention, animal originated cornea is prepared as wound healing material in frozen and dried plates or fluid form. Said wound healing material consisting of animal originated cornea can be effective in processes requiring various tissue repairs such as ophthalmic and non-ophthalmic, skin, mucosa and similar wet skin surface wounds, surgical cuts, skin ulcers, circulation disorder ulcers, diabetic wounds, cancer related wounds, chemical or burn wounds, bonds and tendon wounds in joints, military, sportive injures, skin rejuvenations.

Another purpose of the invention is to exhibit high value added and innovative products by use of animal cornea which is a biological waste status in current case for skin injuries treatment and/or cosmetic aimed skin rejuvenations process.

A further purpose of the invention is to exhibit a wound healing material minimizing late healing, not healing, deepening and/or infected wounds and organ loss arising thereof. Thanks to veinless structure and self-repairing capability of cornea, wounds of high numbers and types can be treated quickly and with no scaring and thus economic and medical problems caused by long treatment processes are eliminated.

Another purpose of the invention is to provide an alternative for amnion membrane used for wound healing in the related art. Thus it provides a raw material which can be used instead of amnion membrane difficult to be supply and having a high cost and offering important technical advantages when compared to the amnion membrane.

A further purpose of the invention is to prepare a dressing and treatment product which has low side effects chance.

The invention also aims to provide an easy to reach, hygienic, standardized and sterile cornea material.

The structural and characteristics features and all advantages of the invention will be understood better with detailed descriptions given below.

### DETAILED DESCRIPTION OF THE INVENTION

In this detailed description, the preferred embodiments of the invention have been described in a manner not forming any restrictive effect and only for purpose of better understanding of the matter.

The invention relates to use of animal originated cornea in treatment of eye and out of eye wounds. Provided that not limited, examples of such wounding are wet skin surface wounds such as skin, mucosa wounds, surgical cuts, skin ulcers, circulation disorder ulcers, diabetic wounds, cancer related wounds, chemical or burn wounds, tissue and tendon wounding in joints, military, sportive wounding, skin rejuvenation processes, intra oral wounds, sick bed wounds, wounds in anus area, post-surgical wounds and scars, tissue repair requiring tissue deficiencies. Animal sources where cornea can be taken (provided that not limited to those given herein) include bovine, ovine and poultry and pig species.

For method of preparing said animal originated cornea as wound healer, firstly, eyeball where cornea tissue is taken is provided and procedures of removing cornea from said cornea sphere are realized. After cornea part removed from eyeball is waited in various solutions and flushed and cleaned, it is transformed into desired product form.

Supply of animal originated cornea tissues is provided from removal of eyeballs under vets control from animal reproduction and slaughtering farms. At this stage, conduct of screening for zoonose (a disease which mess with from an animal to human) and no detection of positive results in terms of particularly Bovine Spongiform (Encephalopathy), Anthrax, tuberculosis and brucellosis are important during production stage and pre-slaughtering. Said obtained eyeballs are kept in preferably with sterile plastic bags and ice blocks ready for processing. During processing of cornea, initially cornea is removed from eyeballs by means of scalpel and flushed by use of balanced saline solution at least three times and tissue particulates and blood remaining in eyeballs are removed. Said balanced saline solution is BSS® Alcon Inc., 6201 South Freeway, Fort Worth., Tex., 76101. Then, corneal buttons taken from eyeball are flushed in antibiotic solution consisting of 50 mcg/ml penicillin, 50 mcg/ml streptomycin, 100 mcg/ml neomycin and 2.5 mcg/ml amphotericin B. Said flushing process can also be made by various chemicals or agents for use of corneal buttons for treatment of various wound types. Thus absorption of various hormones (for instance, growth hormone etc.), polypeptides (for instance insulin), vitamins (for instance Vitamin E a) and some medicines (for instance antibiotics) can be provided.

Cornea removed from eyeball and flushed is waited on a smooth surface (preferably nitrocellulose filter) in a manner outer surface facing down, inner surface contacting eyeball facing up in order to prevent damage on cornea bottom epithelia preventing formation of undesired oedema. Another material not consisting dye, colouring agents, decolorizing agent, colorizing agent, not affected by freezing and unfreezing operations can also be used instead of said filter surface. In another preferred embodiment of the invention, filter paper wherein no bleaching is used as filter surface and bonded by compressing without use of glue can be used. In line with purpose of storage of said cornea, cornea button is hung preferably onto miniature hangers by latch from sides and thus damage to tissue in form of frame named limbos framing outer surface epithelia, inner surface endothelia and cornea circularly and providing surface healing can be prevented. At this stage, if the prepared cornea plates are to be used fresh in 24 hours, they are prepared on filter paper without waiting in any carrying solution, and can be used. Therefore, a hygiene dressing material ready for use in wound areas is obtained.

On the other hand, in case a cornea tissue in plate form not containing in vivo cell is desired for another application (for instance, cases where legal obstacles or tissue rejection probability are high for use of in vivo tissue), said cornea particles are prepared in 0.5×0.5 cm, 0.5×1 cm, 1×2 cm, 1×3, 2×2 cm and 3×3 cm sizes and frozen by means of slow freezing technique and solved at room temperature and then drying process is started. Drying process is performed in a vacuum container at 0.2 Torr pressure and for 24 hours. Then treatment with glutaraldehyde solution of 0.25% is performed and kept at room temperature for one hour. Then, it is flushed with buffered phosphate (PBS) in saline solution and glutaraldehyde wastes are removed. Lastly, dry frozen plates are sterilized by ethylene oxide and prepared in plate product ready for applying onto wound.

Particularly, when cornea tissue in powder form not containing in vivo cell is desired, cornea particulates are frozen in storage solution by slow freezing technique. After unfreezing at room temperature, tissues are dried, crushed in sterile crushing tools into powder form and can be prepared in vials of different volumes from 0.25 cc to 6.0 cc (0.25 cc, 0.50 cc, 1.0 cc, 2.0 cc, 4.0 cc, 6.0 cc).

However, if cornea buttons production in a form wherein cell in vivo is kept is desired, after dehydration process by use of chemicals, tissues are frozen at fluid nitrogen tanks of −80 to −20° C. temperatures by quick freezing techniques and then unfreezing at room temperature before use. Frozen cornea buttons obtained by this method are safe in terms of sterility and can be stored for up to one year and can be shipped long distance.

In conclusion, animal originated cornea tissue are used as described below;
is brought suspension by crushing in order to administer into parts such as intra joints, subcutaneous sections by injector,
is homogenized in cream base in order to use as dressing material in broad wound areas,
is used as plate or homogenates by absorbing as insulin, vitamin, antibiotics, growing hormone, silver, cortisone therapeutics,
is used in serum form by crushing and cleaned of cells for injecting subcutaneous use by too small needles,
as drop in serum form,
in plate form containing in vivo cells by quick freezing technique,
in plate form containing cells losing vitality by slow freezing technique.

1. An animal originated cornea layer for use in a method of treating a wound.

2. A serum comprising the animal originated cornea layer of claim 1.

3. A dressing material comprising the animal originated cornea layer of claim 1.

4. A pharmaceutics composition in cream form comprising the animal originated cornea layer of claim 1.

5. A pharmaceutics composition in suspension form comprising the animal originated cornea layer of claim 1.

6. A homogenate treated with therapeutics comprising the animal originated cornea layer of claim 1.

7. A plate produced by a quick freezing technique and comprising in vivo cells characterized in comprising the animal originated cornea layer of claim 1.

8. A plate produced by slow freezing technique and comprising cells losing vitality characterized in comprising the animal originated cornea layer of claim 1.
